(19) United States
(12) Patent Application Publication
(10) Pub. No.: US 2026/0033910 A1
(43) Pub. Date: Feb. 5, 2026

(54) OPHTHALMIC SYSTEM AND SURGICAL EYE DETERMINATION METHOD

(71) Applicant: TOPCON CORPORATION, Tokyo (JP)

(72) Inventor: Noriyasu TAKEDA, Tokyo (JP)

(73) Assignee: TOPCON CORPORATION, Tokyo (JP)

(21) Appl. No.: 19/223,143

(22) Filed: May 30, 2025

(30) Foreign Application Priority Data
Aug. 1, 2024 (JP) 2024-125936

(51) Int. Cl. A61B 90/00; A61F 9/007; G06V 10/70; G06V 40/18

(52) U.S. Cl. CPC A61B 90/37; A61F 9/007; G06V 10/70; G06V 40/18; A61B 2090/372; G06V 2201/03

(57) ABSTRACT

An ophthalmic system includes: an imaging device configured to capture a body image including at least an upper body of a patient while the face of the patient is covered by a covering sheet having an opening that exposes a surgical eye, the surgical eye being an eyeball to undergo surgery; a processor configured to determine the surgical eye based on surgical eye determination information derived from the body image and preliminary examination surgical eye data of the patient, the preliminary examination surgical eye data having been registered as a result of a preliminary examination; and a notification device configured to notify a surgeon of a result of the determination regarding the surgical eye.

### CROSS-REFERENCE TO RELATED APPLICATION

This application is based on and claims priority under 35 USC 119 from Japanese Patent Application No. 2024-125936, filed on Aug. 1, 2024, the entire content of which is incorporated herein by reference.

### TECHNICAL FIELD

The present disclosure relates to an ophthalmic system and a surgical eye determination method.

### BACKGROUND

A related art discloses surgery for inserting an intraocular insertion medical instrument such as an intraocular lens. In such surgery, there is a risk that an eye to undergo surgery may be mistaken. In order to prevent such a mistake, Patent Literature 1 (Japanese Patent No. 6805581) discloses an ophthalmic information processing device that processes information on a patient eye into which an intraocular insertion medical instrument is to be inserted.

The ophthalmic information processing device acquires patient eye information, which is information created before surgery is performed and is information in which a plurality of pieces of information including information on medical instrument to be inserted and pre-surgery image information are associated with each patient eye. The information on medical instrument to be inserted is information for identifying an intraocular insertion medical instrument to be inserted into the patient eye. The pre-surgery image information is anterior segment image information of the patient eye before surgery. Further, the ophthalmic information processing device acquires intraoperative image information, which is anterior segment image information of the patient eye during surgery, and preparatory medical instrument information for identifying the intraocular insertion medical instrument prepared during surgery. The ophthalmic information processing device compares the pre-surgery image information and the intraoperative image information, specifies the patient eye to undergo surgery, and compares the information on medical instrument to be inserted and the preparatory medical instrument information associated and the patient eye, thereby determining whether the preparation of the surgery is appropriate.

### SUMMARY

However, the ophthalmic information processing device described in Japanese Patent No. 6805581 has the following problems.

In order to acquire intraoperative image information that is the anterior segment image information of the patient eye during surgery, a special imaging device is required to image an anterior segment of the patient immediately before the start of surgery. This imaging not only takes time and effort, but also requires processes such as extracting a characteristic portion by performing a predetermined image process on the captured image. Thus, there is a problem that it requires a lot of time and effort from medical professionals. Therefore, it takes a certain period of time before the surgery can start, which may increase the burden on the patient.

The present disclosure has been made in view of such circumstances, and an object of the present disclosure is to provide an ophthalmic system and a surgical eye determination method capable of efficiently reducing a mistake in identifying a surgical eye of a patient, which is an eye to undergo surgery.

An ophthalmic system includes: an imaging device configured to capture a body image including at least an upper body of a patient while the face of the patient is covered by a covering sheet having an opening that exposes a surgical eye, the surgical eye being an eyeball to undergo surgery; a processor configured to determine the surgical eye based on surgical eye determination information derived from the body image and preliminary examination surgical eye data of the patient, the preliminary examination surgical eye data having been registered as a result of a preliminary examination; and a notification device configured to notify a surgeon of a result of the determination regarding the surgical eye.

According to the present disclosure, it is possible to efficiently reduce a mistake in identifying a surgical eye of a patient, which is an eye to undergo surgery.

### BRIEF DESCRIPTION OF DRAWINGS

FIG. 1 is an overall view of an ophthalmic system according to the present illustrative embodiment.

FIG. 2 is a block diagram showing a hardware configuration example of a server and a terminal device according to the present illustrative embodiment.

FIG. 3 is a diagram showing an arrangement example of a surgical field camera and a surgical field monitor.

FIG. 4 is a diagram showing an example of a drape that covers a patient before surgery.

FIG. 5 is a diagram showing an example of training data used for machine learning.

FIG. 6 is a flowchart showing an example of an operation procedure of the terminal device according to the present illustrative embodiment in chronological order.

### DETAILED DESCRIPTION

Hereinafter, an example of the present disclosure will be described with reference to the drawings, but the scope of the present disclosure is not limited to the illustrative embodiment described here, and various modifications can be made without departing from the scope of the present disclosure.

### 1. Configuration of Ophthalmic System

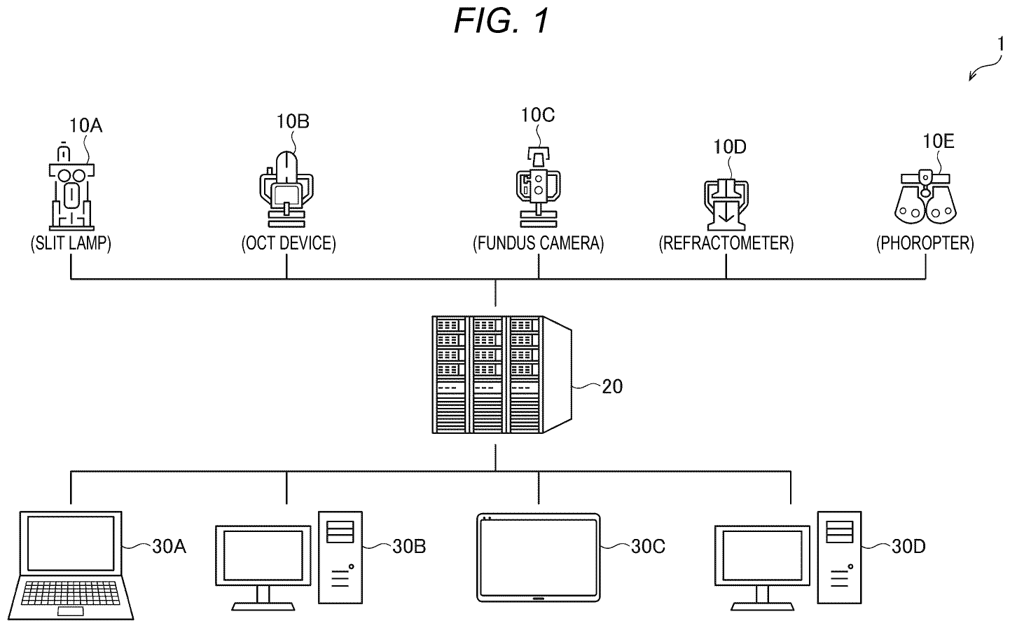

First, a configuration example of an ophthalmic system 1 according to the present illustrative embodiment will be described with reference to FIG. 1.

FIG. 1 is an overall view of the ophthalmic system 1 according to the present illustrative embodiment. The ophthalmic system 1 includes a plurality of ophthalmic devices, a server 20, and a plurality of terminal devices 30A, 30B, 30C, and 30D. Each of the ophthalmic devices or communication devices connected to the ophthalmic devices and the server 20, and each of the terminal devices 30A to 30D and the server 20 are connected via a network. For example, an examination result obtained by the ophthalmic device is stored in the server 20 via the network, so that an examiner using the terminal device (for example, the terminal device 30A) can check the examination result. The number of ophthalmic devices and the number of terminal devices are merely examples, and are not limited.

As shown in FIG. 1, the ophthalmic devices include a slit lamp 10A, an OCT device 10B, a fundus camera 10C, a refractometer 10D, and a phoropter 10E. Each of the ophthalmic devices may be owned by different medical institutions (for example, a general hospital, an ophthalmic clinic, a university hospital, or the like), or a specific medical institution may own a plurality of ophthalmic devices.

For example, the fundus camera 10C is a device owned by many medical institutions, and thus models with different manufacturers and functions are introduced. However, in the ophthalmic system 1, regardless of the manufacturer, the model, or the like, the examiner can check the examination result, including information obtained by other medical institutions, on at least one of the terminal devices 30A to 30D.

Here, features of each ophthalmic device will be briefly described.

The slit lamp 10A, also known as a slit lamp microscope, is a device that obtains an anterior segment image, which is an image of a cross section of a cornea, by irradiating a subject eye, which is an eyeball of a subject, with elongated slit-shaped illumination light from an oblique direction. The slit lamp 10A is used to examine, for example, various anterior segments such as corneal shapes, corneal endothelial cells, and Meibomian glands.

The OCT device 10B is an device that obtains a tomographic image of a fundus (retina) by dividing low-coherence light into two parts, irradiating the fundus with one part, causing backscattered light of the one part to interfere with the other part, and receiving the light with a CCD image sensor or the like. The tomographic image is used to determine eye diseases such as glaucoma, diabetic retinopathy, and retinal pigment degeneration. As the OCT apparatus 10B, a device of any type such as a Fourier domain type, a time domain type, or a swept source type can be used.

The fundus camera 10C is a device that irradiates a fundus portion of the subject eye with illumination light and receives fundus reflected light by a light receiving element such as charge coupled devices (CCD) image sensor to acquire data of a fundus image. The fundus image is used to determine eye diseases such as glaucoma, diabetic retinopathy, and age-related macular degeneration.

The refractometer 10D is an examination device capable of measuring various eye characteristics such as an intraocular pressure value, an eye refractive power, and a corneal curvature of the subject eye.

The phoropter 10E, also known as vision tester, is an examination device used to select a lens suitable for the subject eye. The phoropter 10E includes a plurality of lenses, and by inserting one or a plurality of the lenses on the optical axis, the eye refractive power such as myopia, hyperopia, and astigmatism, or a binocular visual function can be measured.

The ophthalmic device 10 may include devices other than those described above. For example, the ophthalmic device 10 may include a scanning laser ophthalmoscope which performs scanning using a light beam to irradiate the fundus and receives reflected light or fluorescence at a predetermined portion with a CCD or the like to acquire image data of the fundus image. Another example is a specular microscope capable of imaging the corneal endothelial cells, measuring a density, and measuring the corneal thickness.

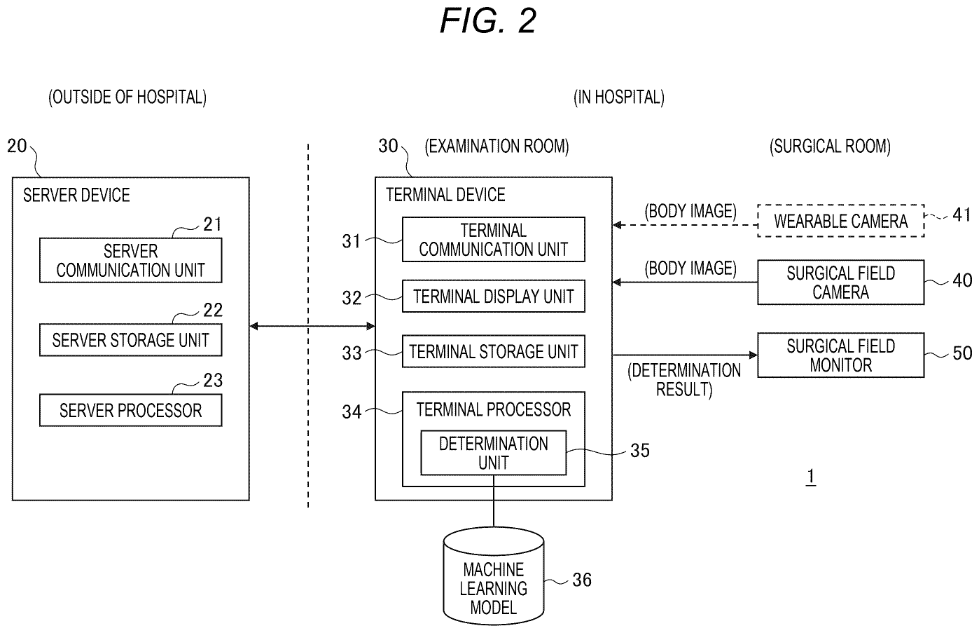

As will be described later with reference to FIG. 2, the server 20 includes an internal server storage unit 22, which stores the examination result, the image data, and the like of the ophthalmic device (see above) transmitted via the network. Further, since each of the terminal devices 30A to 30D is connected to the server 20, the examination result and the like stored in the server storage unit 22 of the server 20 can be accessed at any time. The type of the server 20 is not particularly limited, and may be, for example, a cloud server or an on-premise server disposed in a medical institution or the like.

Each of the terminal devices 30A to 30D is a PC, a notebook PC, a tablet terminal, or the like provided in each medical institution. Software for using the ophthalmic system 1 is installed in each of the terminal devices 30A to 30D. Therefore, the examiner can check, on the terminal devices 30A to 30D, the examination result and the image data stored in the server 20. Further, since the examiner can confirm recorded items of an electronic medical record in addition to the examination results and the like acquired by other medical institutions, the examiner determines the presence or absence of a disease of the subject eye in consideration of each piece of information.

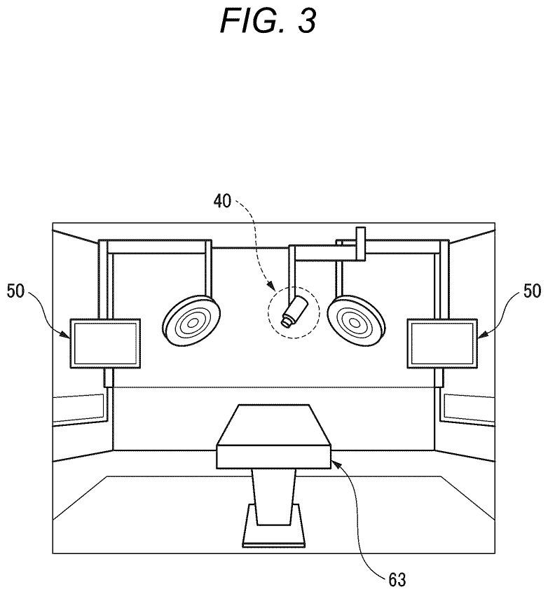

Next, a hardware configuration example of the server 20 and the terminal device 30 constituting the ophthalmic system 1 according to the present illustrative embodiment will be described with reference to FIGS. 2 and 3. FIG. 2 is a block diagram showing a hardware configuration example of the server 20 and the terminal device 30 according to the present illustrative embodiment. FIG. 3 is a diagram showing an arrangement example of a surgical field camera 40 and a surgical field monitor 50. The terminal device 30 has a hardware configuration example applicable to any of the plurality of terminal devices 30A to 30D constituting the ophthalmic system 1 shown in FIG. 1.

The server 20 includes a server communication unit 21, a server storage unit 22, and a server processor 23. The server 20 is disposed outside the medical institution (outside of hospital) and is configured as, for example, a cloud server. Note that the server 20 may be configured as an on-premise server disposed inside a medical institution (in hospital) instead of a cloud server.

The server communication unit 21 is connected to an ophthalmic device (see FIG. 1) disposed inside the medical institution or the terminal device 30 via a network (not shown), and includes a communication circuit that performs data communication with the ophthalmic device or the terminal device 30. The server communication unit 21 performs data communication with the ophthalmic device or the terminal device 30, receives various data transmitted from the ophthalmic device or the terminal device 30, and transmits various data to the ophthalmic device or the terminal device 30.

The server storage unit 22 includes, for example, a random access memory (RAM) and a read only memory (ROM), and temporarily stores a program necessary for the operation of the server 20 and data acquired or generated during the operation. The RAM is, for example, a work memory used during the operation of the server 20. The ROM stores and holds, for example, a program for controlling the server 20 in advance.

The server processor 23 includes, for example, at least one of a central processing unit (CPU), a digital signal processor (DSP), a graphical processing unit (GPU), or a field programmable gate array (FPGA). The server processor 23 functions as a controller that controls the overall operation of the server 20. The server processor 23 performs a control process for controlling the operation of each unit of the server 20, a data input and output process between each unit of the server 20, a data calculation process, and a data storage process. The server processor 23 operates according to the program stored in the server storage unit 22. The server processor 23 uses the server storage unit 22 during operation, and temporarily stores data generated or acquired by the server processor 23 in the server storage unit 22.

The terminal device 30 includes a terminal communication unit 31, a terminal display unit 32, a terminal storage unit 33, and a terminal processor 34. The terminal device 30 is disposed, for example, in an examination room inside a medical institution (in hospital), but the arrangement place is not limited according to an operation policy or the like of the medical institution. The terminal device 30 may be connected to an external storage medium (for example, an SD card) storing a machine learning model 36, and can refer to and execute the machine learning model 36 via the external storage medium. Details of the machine learning model 36 will be described later, but although the machine learning model 36 is exemplified as being stored in an external storage medium, it may also be stored in the terminal storage unit 33 in the terminal device 30 so as to be referred to.

The terminal communication unit 31 is an example of a communication device, and includes a communication circuit that is connected to the server 20 via a network (not shown) and performs data communication with the server 20. The terminal communication unit 31 performs data communication with the server 20, receives various data transmitted from the server 20, and transmits various data to the server 20.

The terminal display unit 32 is an example of a notification device, and displays a result obtained by processing of the terminal processor 34. The terminal display unit 32 is, for example, a display such as a liquid crystal display (LCD) or an organic EL. The terminal display unit 32 may be integrally with an input device (not shown) that receives an operation of a user (for example, a doctor in charge of a patient undergoing surgery), and in this case, the input device and the terminal display unit 32 are integrally as a touch panel display.

The terminal storage unit 33 includes, for example, RAM and ROM, and temporarily stores a program necessary for the operation of the terminal device 30 and data acquired or generated during the operation. The RAM is, for example, a work memory used during the operation of the terminal device 30. The ROM stores and holds, for example, a program for controlling the terminal device 30 in advance.

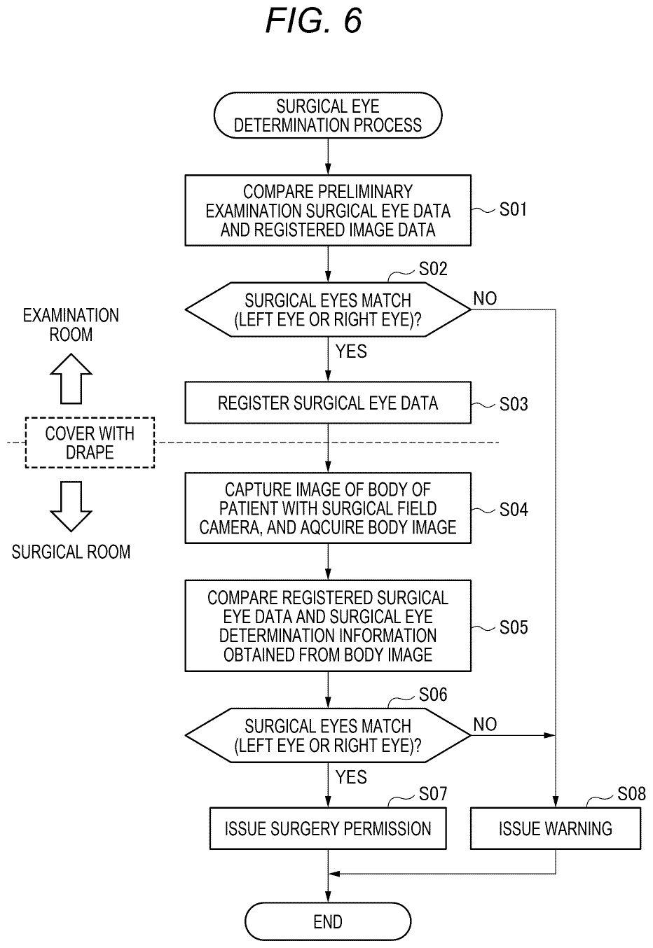

The terminal processor 34 is an example of a processor, and includes at least one of CPU, DSP, GPU, and FPGA, for example. The terminal processor 34 functions as a controller that controls the overall operation of the terminal device 30. The terminal processor 34 performs a control process for controlling the operation of each unit of the terminal device 30, a data input and output process between each unit of the terminal device 30, a data calculation process, and a data storage process. The terminal processor 34 operates according to a program stored in the terminal storage unit 33. The terminal processor 34 uses the terminal storage unit 33 during operation, and temporarily stores data generated or acquired by the terminal processor 34 in the terminal storage unit 33. In the terminal device 30, the terminal processor 34 is configured to execute various processes (for example, the processes in the flowchart shown in FIG. 6) in cooperation with the terminal storage unit 33. The terminal processor 34 cooperates with the terminal storage unit 33 to achieve the functions of a determination unit 35.

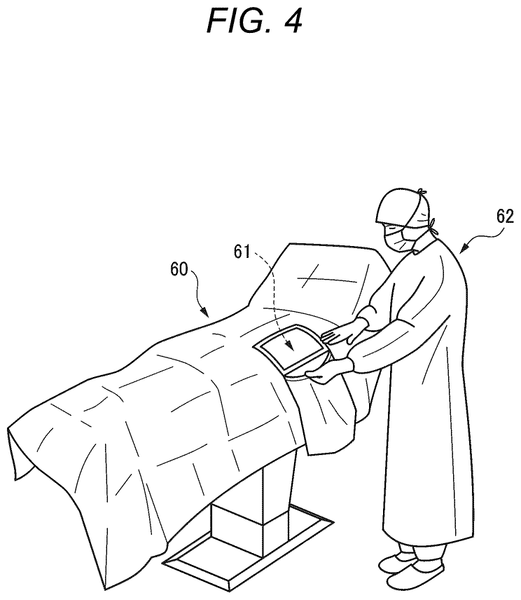

The determination unit 35 inputs a body image of the patient captured by the surgical field camera 40 or a wearable camera 41 immediately before the start of the surgery, and performs a process of specifying surgical eye determination information from the input body image of the patient by referring to the machine learning model 36. The surgical eye determination information includes, for example, not only identification information such as the name of the patient, but also information for specifying whether an eye to undergo surgery (that is, the surgical eye) is the left eye, the right eye, or both eyes of the patient. The determination unit 35 acquires from the server 20 preliminary examination surgical eye data of the patient that is registered as a result of a preliminary examination performed in an examination room or the like before the surgery. The determination unit 35 performs a process of determining the surgical eye of the patient based on the acquired preliminary examination surgical eye data of the patient and the specified surgical eye determination information. Here, the determination of the surgical eye of the patient refers to a process of determining, for example, for a patient covered by the drape 60 (see FIG. 4) since the surgery is about to be started, whether the surgical eye indicated by the surgical eye determination information matches the surgical eye indicated by the preliminary examination surgical eye data of the patient registered as a result of the preliminary examination, either the left eye, the right eye, or both eyes. The determination unit 35 may display a determination result of the surgical eye on the terminal display unit 32.

The machine learning model 36 inputs a body image of the patient captured by the surgical field camera 40 or the wearable camera 41 immediately before the start of the surgery, and performs a process of specifying the surgical eye determination information from the body image of the patient, and outputting the information. The machine learning model 36 is stored in advance in an external storage medium such as an SD card, for example, and is referred to and used by the determination unit 35 after the external storage medium is connected to the terminal device 30 so as to enable data input and output.

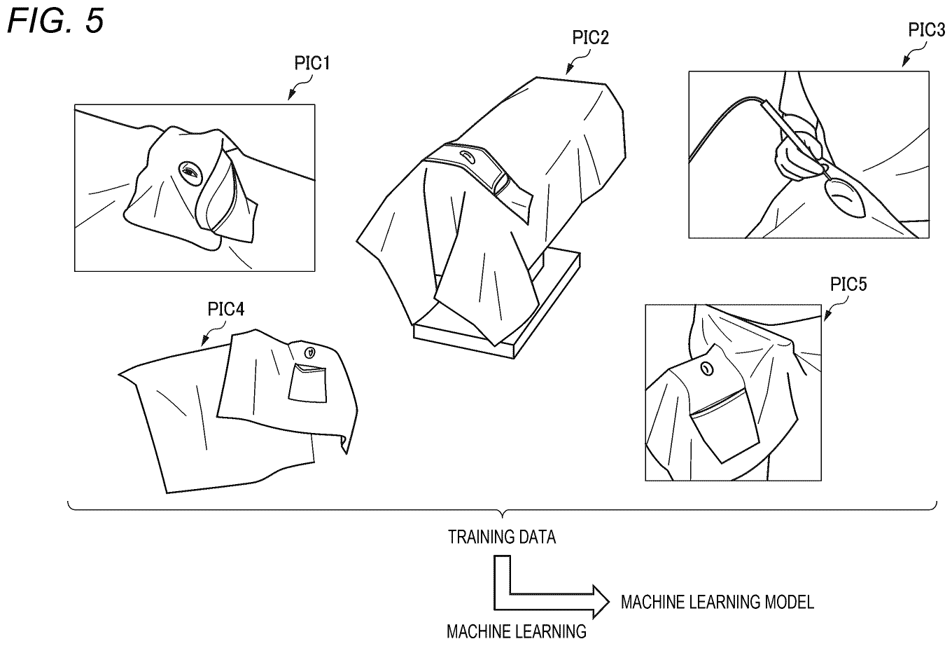

Here, the machine learning of the machine learning model 36 will be described with reference to FIGS. 4 and 5. FIG. 4 is a diagram showing an example of the drape that covers a patient before the surgery. FIG. 5 is a diagram showing an example of training data used for the machine learning. The machine learning model 36 is generated by machine learning using images of a plurality of types of covering sheets and images of a plurality of types of covering sheets covering the face of the patient as training data. For example, as shown in FIG. 4, the covering sheet is the drape 60 or gauze for covering a patient while surgery is being performed on the patient by a surgeon 62. Whether the drape or the gauze is used as the covering sheet may be determined based on the regional property of the medical institution.

The drape 60 shown in FIG. 4 has an opening 61 through which the surgical eye, which is an eye to undergo surgery of the patient, can be exposed. By providing the opening 61, the surgeon 62 can perform an appropriate surgery on the surgical eye in a state where at least the upper body or the whole body of the patient is covered with the drape 60. The state of the drape 60 during surgery is that it has an opening 61 on either the left or right side. That is, it is assumed that there is no drape that has an opening through which both left and right eyes are exposed or an opening that connects the left and right sides.

As shown in FIG. 5, input images serving as the training data include input images PIC1, PIC2, PIC3, PIC4, and PIC5, but it is needless to say that the input images are not limited to these input images.

The input image PIC1 shows a state in which at least the face of the patient is covered with a drape capable of exposing the left eye of the patient, and the drape has an opening that exposes the left eye of the patient. The input image PIC2 shows a patient lying on a surgical table 63 with a drape placed over the surgical table 63 and the patient, and the drape has an opening that exposes the right eye of the patient. The input image PIC3 shows a state in which at least the face of the patient is covered with a drape capable of exposing the left eye of the patient during surgery, and the drape has an opening that exposes the left eye of the patient. The input image PIC4 shows a drape itself having an opening capable of exposing the left eye of the patient. The input image PIC5 shows a state in which the whole body including at least the face of the patient is covered with a drape capable of exposing the right eye of the patient immediately before the surgery, and the drape has an opening that exposes the right eye of the patient.

The machine learning model 36 is generated by performing machine learning using such a large number of input images PIC1 to PIC5 as training data. The machine learning model 36 is appropriately referred to and executed by the determination unit 35 of the terminal processor 34 of the terminal device 30. It is assumed that, during surgery, the patient is lying supine on the surgical table 63 (see FIG. 3) installed in a surgical room. As a result, if the upper side of the head of the patient can be recognized, it is determined whether the exposed eyeball of the patient is left or right.

The surgical field camera 40 is an example of an imaging device, is disposed, for example, in a surgical room inside a medical institution (in hospital) (see FIG. 3), and is connected to the terminal device 30 so as to enable data communication. The surgical field camera 40 captures an image of the surround with the surgical table 63 as a main subject. For example, in a state where a patient is lying on the surgical table 63 with at least the upper body or the whole body covered with a covering sheet such as the drape 60 immediately before surgery, the surgical field camera 40 captures a body image including at least the upper body of the patient. The surgical field camera 40 transmits the captured body image of the patient to the terminal device 30.

The wearable camera 41 is an example of the imaging device, for example, is a small camera attached to a surgeon such as a doctor or an assistant who performs the surgery in the surgical room, and is connected to the terminal device 30 so as to enable data communication similarly to the surgical field camera 40. The wearable camera 41 captures an image of the surround with the surgical table 63 as a main subject. For example, in a state where a patient is lying on the surgical table 63 with at least the upper body or the whole body covered with a covering sheet such as the drape 60 immediately before surgery, the wearable camera 41 captures a body image including at least the upper body of the patient as viewed from the position of the surgeon. The wearable camera 41 transmits the captured body image of the patient to the terminal device 30.

The surgical field monitor 50 is an example of the notification device, is disposed, for example, in a surgical room inside a medical institution (in hospital) (see FIG. 3), and displays various types of information or data necessary for surgery. For example, the surgical field monitor 50 may display a result of a series of processes (see FIG. 6) performed by the terminal device 30. By visually checking the information or data displayed on the surgical field monitor 50, the surgeon can grasp various pieces of information on the patient and on the surgical eye of the patient.

### 2. Operation Procedure of Ophthalmic System (Surgical Eye Determination Method)

Next, an example of an operation procedure of the ophthalmic system 1 according to the present illustrative embodiment will be described with reference to FIG. 6. FIG. 6 is a flowchart showing an example of an operation procedure of the terminal device 30 according to the present illustrative embodiment in chronological order. A series of processes illustrated in FIG. 6 is mainly executed by the terminal processor 34 of the terminal device 30. As illustrated in FIG. 6, in the ophthalmic system 1 according to the present illustrative embodiment, the terminal device 30 performs a double check in which the process of determining whether the surgical eye of the patient (left or right) matches is performed twice in total, a first determination in step S02 and a second determination in step S06.

In FIG. 6, the terminal processor 34 acquires the preliminary examination surgical eye data, which is the surgical eye data acquired in the preliminary examination performed before the surgery for the patient who is the subject of surgery, from the server 20 or by reading it from the terminal storage unit 33 (S01). The terminal processor 34 acquires from the server 20 past surgical eye data, which is the surgical eye data acquired in the past and stored in the server 20 in association with the same patient (S01). The terminal processor 34 compares the acquired preliminary examination surgical eye data with the past surgical eye data (S01). Since there is a high possibility that some disease can be confirmed in a surgical eye, the preliminary examination surgical eye data and the past surgical eye data may be at least one of, for example, an anterior segment image which is an image of a cross section of a cornea, a tomographic image of a fundus (retina), a fundus image showing a retina, an optic nerve, or a blood vessel, an optical coherence tomographic blood vessel image for evaluating a state of a blood vessel in a retina or a macular portion, or the like.

In this comparison, the terminal processor 34 may display the acquired preliminary examination surgical eye data and the acquired past surgical eye data on the terminal display unit 32 in a comparable manner, and prompt a surgeon such as a doctor in charge of the surgery to input data and accept the input. Alternatively, the terminal processor 34 may perform image analysis on each of the acquired preliminary examination surgical eye data and past surgical eye data to determine whether the similarity of feature points on respective images of the two types of data is equal to or greater than a predetermined threshold value, thereby performing automatic comparison that does not require input by the surgeon.

In a case where it is determined that the surgical eye of the patient indicated by the preliminary examination surgical eye data does not match the surgical eye of the patient indicated by the past surgical eye data in terms of the left eye, the right eye, or both eyes (S02, NO), the terminal processor 34 generates a warning screen (not illustrated) for the surgeon and notifies the surgeon with the terminal display unit 32 (S08). The warning notifies the surgeon that, for example, the surgical eye of the patient indicated by the preliminary examination surgical eye data does not match the surgical eye of the patient indicated by the past surgical eye data in terms of the left eye or the right eye, and also notifies the surgeon that it is better not to start the surgery.

On the other hand, in a case where it is determined that the surgical eye of the patient indicated by the preliminary examination surgical eye data matches the surgical eye of the patient indicated by the past surgical eye data in terms of the left eye, the right eye, or both eyes (S02, YES), the terminal processor 34 registers the preliminary examination surgical eye data as surgical eye data before surgery officially (S03). After the process of step S03, since it is officially determined whether the surgical eye is the left eye or the right eye, a covering sheet (for example, the drape 60) is placed over the patient who is the subject of surgery, and the patient is carried into the surgical room with the drape 60 on.

The terminal processor 34 acquires a body image which is an image of the upper body or the whole body including at least the face of the patient captured by the surgical field camera 40 disposed in the surgical room (S04). The terminal processor 34 inputs the body image of the patient immediately before the start of the surgery acquired in step S04, and performs a process of specifying the surgical eye determination information from the input body image of the patient by referring to the machine learning model 36 (S05). Further, the terminal processor 34 acquires the preliminary examination surgical eye data of the patient registered as the result of the preliminary examination registered in step S03, and performs a process of determining the surgical eye of the patient based on a comparison between the acquired preliminary examination surgical eye data of the patient and the surgical eye determination information specified in step S05 (S06).

In a case where it is determined that the surgical eye of the patient indicated by the preliminary examination surgical eye data does not match the surgical eye of the patient indicated by the specified surgical eye determination information in terms of the left eye, the right eye, or both eyes (S06, NO), the terminal processor 34 generates a warning screen (not illustrated) for the surgeon and notifies the surgeon with the terminal display unit 32 (S08). This warning is displayed at least on the surgical field monitor 50, so that the surgeon in the surgical room can check the warning.

On the other hand, in a case where it is determined that the surgical eye of the patient indicated by the preliminary examination surgical eye data matches the surgical eye of the patient indicated by the specified surgical eye determination information in terms of the left eye, the right eye, or both eyes (S06, YES), the terminal processor 34 displays a notification on the terminal display unit 32 or the surgical field monitor 50 to inform the surgeon that permission to start the surgery is granted (S07). Accordingly, the surgeon can confirm that the surgical eye of the patient exposed through the opening of the drape 60 is correct, and thus can start the surgery appropriately.

As described above, in the ophthalmic system, whether the surgical eye is the left eye or the right eye is determined using the pre-surgery determination information obtained from the body image captured by the imaging device at a timing before the start of the surgery, and the preliminary examination surgical eye data of the patient registered as the result of the preliminary examination performed before the surgery. Then, the notification device notifies the surgeon of the determination result. As a result, according to the ophthalmic system, it is possible to efficiently reduce a mistake in identifying a surgical eye of a patient, which is an eye to undergo surgery.

Other aspects and effects of the present disclosure are as follows. In another aspect of the ophthalmic system according to the present disclosure, the processor inputs an image in which the covering sheet covers the face of a patient, and determines the surgical eye by referring to a machine learning model that is subject to machine learning to output whether the left or right eyeball of the patient is exposed through the opening of the covering sheet.

Further, in the ophthalmic system, the processor determines the surgical eye by referring to the machine learning model. The machine learning model is generated by machine learning in which an image of the covering sheet covering the face of the patient is input and whether the left or right eyeball of the patient is exposed from the opening of the covering sheet is output. As a result, according to the ophthalmic system, it is possible to accurately determine whether the surgical eye is the left eye or the right eye of the patient using the machine learning model generated by machine learning before surgery.

In another aspect of the ophthalmic system according to the present disclosure, the machine learning model is generated by machine learning using, as training data, images of a plurality of types of the covering sheets and images of a plurality of types of the covering sheets covering the face of a patient.

In the ophthalmic system, not only images of a plurality of types of covering sheets, but also images of a plurality of types of the covering sheets covering the face of a patient are adopted as training data when the machine learning model is subject to the machine learning. As a result, according to the ophthalmic system, it is possible to detect the covering sheet covering the patient with high accuracy before surgery by the machine learning model, and a surgeon can accurately grasp whether the surgical eye of the patient is the left eye or the right eye through the opening of the covering sheet.

In another aspect of the ophthalmic system according to the present disclosure, the imaging device is a surgical field camera disposed in a surgical room where surgery is performed or a wearable camera attached to the surgeon for surgery.

In the ophthalmic system, the imaging device may be a commonly available surgical field camera disposed in advance in a surgical room where the surgery is performed, or a camera that the surgeon already wears for surgical procedures. Since such a commonly available camera can be used, there is no need for a special imaging device for imaging the surgical eye of the patient before the surgery in accordance with the surgery. Further, since it is not necessary to perform image analysis on an image captured by such an imaging device, it is possible to execute the surgery promptly without the patient waiting for the start of surgery more than necessary.

An aspect of the terminal device according to the present disclosure further includes a communication device configured to perform data communication with a server storing past surgical eye data, which is data on the surgical eye of the patient acquired in past, wherein the processor compares, for the surgical eye of the same patient, the preliminary examination surgical eye data and the past surgical eye data acquired from the server via the communication device.

Further, in the ophthalmic system, the processor compares the preliminary examination surgical eye data and the past surgical eye data acquired from the server storing the past surgical eye data, which is data on the surgical eye of the patient acquired in the past. As a result, according to the ophthalmic system, it is possible to grasp before the surgery whether the surgical eye of the patient indicated by the preliminary examination surgical eye data matches the surgical eye of the patient indicated by the past surgical eye data.

In another aspect of the terminal device according to the present disclosure, the processor is configured to, when the preliminary examination surgical eye data matches the past surgical eye data as a result of the comparison, determine the surgical eye using the surgical eye determination information and the preliminary examination surgical eye data.

Further, in the ophthalmic system, when the preliminary examination surgical eye data matches the past surgical eye data, the processor determines the surgical eye of the patient using the surgical eye determination information and the preliminary examination surgical eye data. As a result, according to the ophthalmic system, it is possible to effectively prevent a mistake in identifying the surgical eye of the patient prior to the surgery through double-checking, which includes a first comparison between the preliminary examination surgical eye data and the past surgical eye data, and a second comparison between the surgical eye determination information and the preliminary examination surgical eye data.

In another aspect of the terminal device according to the present disclosure, the processor is configured to, when a surgical eye of the patient indicated by the surgical eye determination information obtained from the body image does not match a surgical eye of the patient indicated by the preliminary examination surgical eye data of the patient registered as the result of the preliminary examination, issue a pre-surgery warning with the notification device.

In the ophthalmic system, when a surgical eye of the patient indicated by the surgical eye determination information obtained from the body image does not match a surgical eye of the patient indicated by the preliminary examination surgical eye data of the patient registered as the result of the preliminary examination, the processor issues a pre-surgery warning with the notification device. As a result, according to the ophthalmic system, in a case where a surgeon mistakenly identifies the surgical eye of the patient, even though mistakes like this should never happen, it is possible to prompt the surgeon to redo the preparation for the surgery by the pre-surgery warning.

In another aspect of the terminal device according to the present disclosure, the processor is configured to, when a surgical eye of the patient indicated by the surgical eye determination information obtained from the body image matches a surgical eye of the patient indicated by the preliminary examination surgical eye data of the patient registered as the result of the preliminary examination, issue a surgery permission with the notification device.

In the ophthalmic system, when a surgical eye of the patient indicated by the surgical eye determination information obtained from the body image matches a surgical eye of the patient indicated by the preliminary examination surgical eye data of the patient registered as the result of the preliminary examination, the processor issues a surgery permission with the notification device. As a result, according to the ophthalmic system, whether the surgical eye of the patient is the left eye, the right eye, or both eyes is correctly recognized by the surgeon based on the determination of the preliminary examination surgical eye data and the surgical eye determination information obtained immediately before the surgery, so that the surgery can be safely started.

Another aspect of the present disclosure provides a surgical eye determination method including: for a surgical eye which is an eyeball to undergo surgery, confirming the surgical eye by performing comparison between preliminary examination surgical eye data of a patient obtained as a result of a preliminary examination and past surgical eye data, which is data on the surgical eye of the patient acquired in past; capturing a body image including at least an upper body of the patient while the face of the patient is covered with a covering sheet having an opening; determining the surgical eye based on surgical eye determination information obtained from the body image and the preliminary examination surgical eye data of the patient determined to match the past surgical eye data as a result of the comparison; and notifying a surgeon of a determination result of the surgical eye.

In the surgical eye determination method, when the preliminary examination surgical eye data of the patient registered as a result of the preliminary examination performed before the surgery matches the past surgical eye data related to the surgical eye of the same patient acquired in the past, the surgical eye of the patient is determined using the surgical eye determination information and the preliminary examination surgical eye data. In this determination, it is determined whether the surgical eye is the left eye or the right eye, and the surgeon is notified of the determination result. As a result, according to the surgical eye determination method, it is possible to effectively prevent a mistake in identifying the surgical eye of the patient before the surgery by double checking including a first comparison between the preliminary examination surgical eye data and the past surgical eye data, and a second comparison between the surgical eye determination information and the preliminary examination surgical eye data.

The present disclosure is not limited to the illustrative embodiment described above and may be implemented in various modes without departing from the gist thereof.

The whole or part of the illustrative embodiments disclosed above can be described as, but not limited to, the following supplementary notes.

1. An ophthalmic system including:
an imaging device configured to capture a body image including at least an upper body of a patient while the face of the patient is covered by a covering sheet having an opening that exposes a surgical eye, the surgical eye being an eyeball to undergo surgery;
a processor configured to determine the surgical eye based on surgical eye determination information derived from the body image and preliminary examination surgical eye data of the patient, the preliminary examination surgical eye data having been registered as a result of a preliminary examination; and
a notification device configured to notify a surgeon of a result of the determination regarding the surgical eye.

2. The ophthalmic system according to 1,
wherein the processor is configured to input an image in which the covering sheet covers the face of a patient, and to determine the surgical eye by referring to a machine learning model trained using machine learning to identify whether the left or right eyeball of the patient is exposed through the opening of the covering sheet.

3. The ophthalmic system according to 2,
wherein the machine learning model is generated by machine learning using, as training data, images of a plurality of types of the covering sheets, and images of a plurality of types of the covering sheets covering the face of a patient.

4. The ophthalmic system according to 1,
wherein the imaging device is either a surgical field camera disposed in a surgical room where surgery is performed or a wearable camera attached to the surgeon for surgery.

5. The ophthalmic system according to 1, further including:
a communication device configured to perform data communication with a server storing past surgical eye data, the past surgical eye data being data on the surgical eye of the patient acquired in the past,
wherein the processor is configured to compare, for the surgical eye of the same patient, the preliminary examination surgical eye data and the past surgical eye data acquired from the server via the communication device.

6. The ophthalmic system according to 5,
wherein the processor is configured to determine the surgical eye using the surgical eye determination information and the preliminary examination surgical eye data, based on a match between the preliminary examination surgical eye data and the past surgical eye data as a result of the comparison.

7. The ophthalmic system according to 1,
wherein the processor is configured to issue a pre-surgery warning via the notification device in a case where the surgical eye of the patient indicated by the surgical eye determination information obtained from the body image does not match the surgical eye of the patient indicated by the preliminary examination surgical eye data of the patient registered as the result of the preliminary examination.

8. The ophthalmic system according to 1,
wherein the processor is configured to issue a surgery permission via the notification device in a case where a surgical eye of the patient indicated by the surgical eye determination information obtained from the body image matches the surgical eye of the patient indicated by the preliminary examination surgical eye data of the patient registered as the result of the preliminary examination.

9. A surgical eye determination method including:
confirming a surgical eye, which is an eyeball to undergo surgery, by performing a comparison between preliminary examination surgical eye data of a patient obtained as a result of a preliminary examination and past surgical eye data, which is data for the surgical eye of the patient acquired in the past;
capturing a body image including at least an upper body of the patient while the face of the patient is covered by a covering sheet having an opening;
determining the surgical eye based on surgical eye determination information obtained from the body image and the preliminary examination surgical eye data of the patient determined to match the past surgical eye data as a result of the comparison; and
notifying a surgeon of a result of the determination regarding the surgical eye.

What is claimed is:

1. An ophthalmic system comprising:
an imaging device configured to capture a body image including at least an upper body of a patient while the face of the patient is covered by a covering sheet having an opening that exposes a surgical eye, the surgical eye being an eyeball to undergo surgery;
a processor configured to determine the surgical eye based on surgical eye determination information derived from the body image and preliminary examination surgical eye data of the patient, the preliminary examination surgical eye data having been registered as a result of a preliminary examination; and
a notification device configured to notify a surgeon of a result of the determination regarding the surgical eye.

2. The ophthalmic system according to claim 1,
wherein the processor is configured to input an image in which the covering sheet covers the face of a patient, and to determine the surgical eye by referring to a machine learning model trained using machine learning to identify whether the left or right eyeball of the patient is exposed through the opening of the covering sheet.

3. The ophthalmic system according to claim 2,
wherein the machine learning model is generated by machine learning using, as training data, images of a plurality of types of the covering sheets, and images of a plurality of types of the covering sheets covering the face of a patient.

4. The ophthalmic system according to claim 1,
wherein the imaging device is either a surgical field camera disposed in a surgical room where surgery is performed or a wearable camera attached to the surgeon for surgery.

5. The ophthalmic system according to claim 1, further comprising:
a communication device configured to perform data communication with a server storing past surgical eye data, the past surgical eye data being data on the surgical eye of the patient acquired in the past,
wherein the processor is configured to compare, for the surgical eye of the same patient, the preliminary examination surgical eye data and the past surgical eye data acquired from the server via the communication device.

6. The ophthalmic system according to claim 5,
wherein the processor is configured to determine the surgical eye using the surgical eye determination information and the preliminary examination surgical eye data, based on a match between the preliminary examination surgical eye data and the past surgical eye data as a result of the comparison.

7. The ophthalmic system according to claim 1,
wherein the processor is configured to issue a pre-surgery warning via the notification device in a case where the surgical eye of the patient indicated by the surgical eye determination information obtained from the body image does not match the surgical eye of the patient indicated by the preliminary examination surgical eye data of the patient registered as the result of the preliminary examination.

8. The ophthalmic system according to claim 1,
wherein the processor is configured to issue a surgery permission via the notification device in a case where a surgical eye of the patient indicated by the surgical eye determination information obtained from the body image matches the surgical eye of the patient indicated by the preliminary examination surgical eye data of the patient registered as the result of the preliminary examination.

9. A surgical eye determination method comprising:
confirming a surgical eye, which is an eyeball to undergo surgery, by performing a comparison between preliminary examination surgical eye data of a patient obtained as a result of a preliminary examination and past surgical eye data, which is data for the surgical eye of the patient acquired in the past;
capturing a body image including at least an upper body of the patient while the face of the patient is covered by a covering sheet having an opening;
determining the surgical eye based on surgical eye determination information obtained from the body image and the preliminary examination surgical eye data of the patient determined to match the past surgical eye data as a result of the comparison; and
notifying a surgeon of a result of the determination regarding the surgical eye.
